(19) United States
(12) Patent Application Publication
(10) Pub. No.: US 2016/0176915 A1
(43) Pub. Date: Jun. 23, 2016

(54) PROCESS FOR PREPARATION OF ABIRATERONE ACETATE

(71) Applicant: SUN PHARMACEUTICAL INDUSTRIES LIMITED, Andheri (east), Mumbai (IN)

(72) Inventors: Rajamannar Thennati, Gujarat (IN); Rajeev Budhdev Rehani, Baroda (IN); Nischalkumar Vinodbhai Patel, Baroda (IN); Riteshkumar Rajnikant Kansara, Baroda (IN)

(21) Appl. No.: 14/392,210

(22) PCT Filed: Jun. 24, 2014
(86) PCT No.: PCT/IN2014/000420

§ 371 Date: Dec. 23, 2015

(30) Foreign Application Priority Data
Jun. 24, 2013 (IN) 2133/MUM/2013

(51) Int. Cl. C07J 43/00; C07J 1/00

(52) U.S. Cl. CPC C07J 43/003; C07J 1/0003

(57) ABSTRACT

The present invention relates to improvement in the process of preparation of abiraterone acetate or a pharmaceutically acceptable salt thereof wherein the improvement comprises purifying the crude 3-&-acetoxyandrosta-5,16-diene-17-yl trifluoromethane sulphonate by crystallization from a solvent to obtain acetoxyandrosta-5,16-diene-17-yl trifluoromethane sulphonate as a crystalline solid and converting it to abiraterone acetate or pharmaceutically acceptable salt thereof.

The present invention relates to improvement in the process of preparation of abiraterone acetate or a pharmaceutically acceptable salt thereof wherein the improvement comprises purifying the crude 3-β-acetoxyandrosta-5,16-diene-17-yl trifluoromethane sulphonate by crystallization from a solvent to obtain acetoxyandrosta-5,16-diene-17-yl trifluoromethane sulphonate as a crystalline solid and converting it to abiraterone acetate or pharmaceutically acceptable salt thereof.

### BACKGROUND OF THE INVENTION

Abiraterone acetate of Formula I:

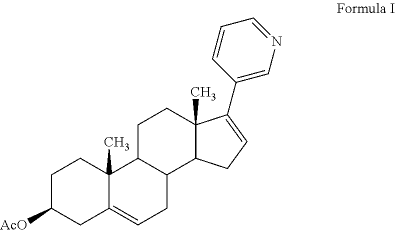

is a potent selective, orally active inhibitor of the key enzyme in testosterone synthesis, 17α-hydroxylase-C17,20-lyase, also known as steroid 17α-monooxygenase inhibitor or Human Cytochrome P450_{17α}. Suppression of testosterone synthesis has been demonstrated with abiraterone acetate in patients with prostate cancer.

The compound was first disclosed in WO-A-93/20097, with a further synthetic method to the compound in WO-A-95/09178 (both British Technology Group Limited). In particular, WO-A-95/09178 discloses the synthesis of a compound of Formula:

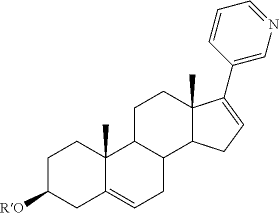

where the 3β substituent R′ is hydrogen or a lower acyl group having 2 to 4 carbon atoms. One of the methods disclosed makes this from the corresponding ketone via the steroidal enol triflate (trifluoromethylsulfonate):

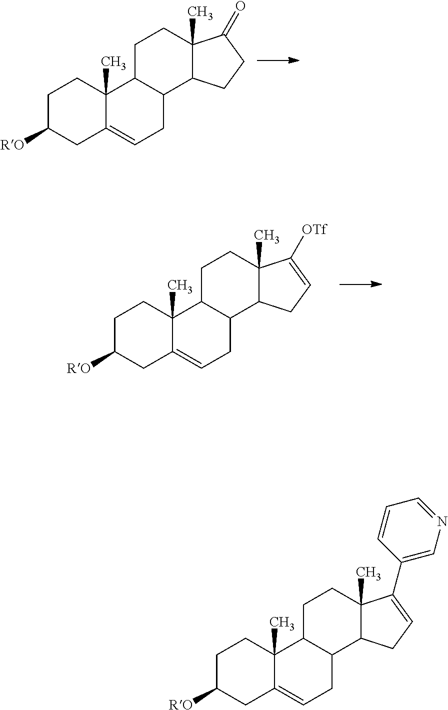

The base used in the reported route, 2,6-di-tert-butyl-4-methylpyridine (DTBMP), is expensive. In prior art it is disclosed that when R′ is a lower acyl group, elimination of the acid occurs, giving an undesirable by-product of formula:

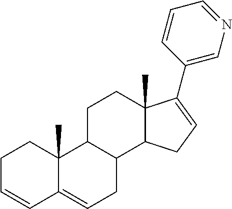 [SMILES: C[C@]12CCC=CC1=CCC1C2CC[C@]2(C)C(c3cccnc3)=CCC12]

This by-product cannot be removed by recrystallization at either step (triflating and final step). Therefore column chromatography is required at both steps.

The triflate intermediate is also reported in J. Med. Chem. (1995), 38(13), 2463-71 (Potter et al.); J. Med. Chem. (1997), 40(20), 3297-3304 (Ling et al.); J. Med. Chem. (2000), 43(22), 4266-4277 (Hartmann et al.); Journal für Praktische Chemie/Chemiker-Zeitung (1993), 335(5), 439-44 (Schweder et al.); Tet. Lett. (1990), 31(13), 1889-1892 and Tet. Lett. (1991), 32(12), 1579-82 (both Ciattini et al.); Archiv der Pharmazie (Weinheim, Germany) (2001), 334(12), 373-374 and Steroid Biochem. Molec. Biol. (2003), 84, 555-562 (both Haidar et al.); Synthesis (1986), 320-322 (Cacchi et al.); and J. Organomet. Chem. (1989), 367(3), 375-82 and Synth. Commun. (1987), 17(12), 1389-402 (both Orsini et al.). All of these references prepare the triflate intermediate according to the method recommended in a review entitled “Perfluoroalkanesulfonic Esters: Methods of Preparation and Application in Organic Chemistry”, Synthesis, 1982, 85-126 (Stang et al.).

Stange et at advocates against the use of simple bases such as pyridine, lutidine or triethylamine; as these give undesirable by-products at the triflate stage. Stang et al. recommend the use of hindered base ie: 2,6-di-tert-butyl-4-methylpyridine (DTBMP) instead, in spite of that fact that this base is expensive.

WO-A-95/09178 suggests replacing the triflate with a corresponding vinyl iodide intermediate, and uses this to make compounds by reacting this with a (3-pyridyl)-substituted borane of Formula:

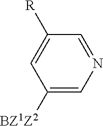

wherein R represents a hydrogen atom or an alkyl group of 1-4 carbon atoms and Z¹ and Z² independently represent hydroxy or alkoxy or alkyl of 1-3 carbon atoms each or Z¹ and Z² together represent an alkylenedioxy group of 2 or 3 carbon atoms. However, column chromatography is required for this process also.

The process for preparation of abiraterone acetate from dehydroepiandrosterone-3-acetate via 3-β-acetoxyandrosta-5,16-diene-17-yl trifluoromethane sulphonate is disclosed in prior arts. For instance, U.S. Pat. No. 7,700,766; Chinese patent applications CN102558274, CN102731605, CN103059090, CN103193849 and CN102030798 disclose preparation of abiraterone acetate starting from dehydroepiandrosterone-3-acetate, converting it to 3-β-acetoxyandrosta-5,16-diene-17-yl trifluoromethane sulphonate followed by coupling with diethyl-(3-pyridyl)-borane in presence of a Palladium catalyst. In all the references 3-β-acetoxyandrosta-5,16-diene-17-yl trifluoromethane sulphonate is isolated as crude form (mostly in form of oil) and is directly taken for next step without subjecting it to purification. The crude oil contains unreacted starting material DHEAA along with other decomposed products. Handling of such oily intermediate on a commercial scale has inherent disadvantages of storage, transport, charging etc J. Med. Chem. (1995), 38(13), 2463-71 (Potter et al.) discloses similar process wherein the 3-β-acetoxyandrosta-5,16-diene-17-yl trifluoromethane sulphonate is subjected to purification by chromatographic techniques viz HPLC. Use of such techniques is certainly not preferred on a commercial manufacturing, particularly in this case where the product is required to be produced on a tonnage scale. Such technique of purification adversely impact the overall productivity thus rendering it expensive and laborious

Thus, there is an unmet need for simple and economical process which can be practiced at commercial scale on a tonnage level, for preparation of 3-β-acetoxyandrosta-5,16-diene-17-yl trifluoromethane sulphonate and Abiraterone acetate of consistent quality, with ease

### SUMMARY OF INVENTION

The present invention provides an improvement in the process of preparation of abiraterone acetate or a pharmaceutically acceptable salt thereof which process comprises a first step in which dehydroepiandrosterone-3-acetate (DHEAA) of Formula II

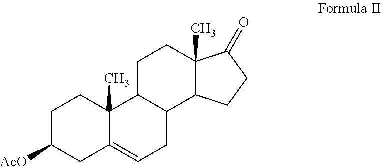

is triflated in the presence of a base and a solvent to form crude 3-β-acetoxyandrosta-5,16-diene-17-yl trifluoromethane sulphonate (Formula III), which is also referred as the triflate intermediate in the present specification,

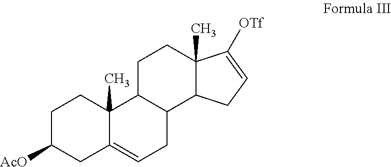

and a second step wherein the crude 3-β-acetoxyandrosta-5,16-diene-17-yl trifluoromethane sulphonate is converted to abiraterone acetate or a salt thereof, wherein the improvement comprises an intermediate step of purifying the crude 3-β-acetoxyandrosta-5,16-diene-17-yl trifluoromethane sulphonate by crystallization from a solvent to obtain acetoxyandrosta-5,16-diene-17-yl trifluoromethane sulphonate as a discrete crystalline solid.

The previous workers have not envisaged preparation of crystalline solid of the triflate intermediate by simple crystallization. Present invention provides a purification process by crystallization to yield a high purity discrete solid triflate intermediate without the need for any chromatographic purification. This discrete solid triflate intermediate has significant impact in achieving a superior purity and a pharmaceutically elegant color of abiraterone acetate besides other desired critical quality attributes as per ICH guidelines, thus obviating any chromatography techniques of purification taught in prior art.

### DETAILED DESCRIPTION OF THE INVENTION

The present invention provides a simple, economical and commercially scalable process for preparation of Abiraterone acetate or pharmaceutically acceptable salts thereof.

According to the present invention there is provided an improvement in the process of preparation of abiraterone acetate or a pharmaceutically acceptable salt thereof which process comprises a first step in which dehydroepiandrosterone-3-acetate (DHEAA) is triflated in the presence of a base and a solvent to form crude 3-β-acetoxyandrosta-5,16-diene-17-yl trifluoromethane sulphonate and a second step wherein the crude 3-β-acetoxyandrosta-5,16-diene-17-yl trifluoromethane sulphonate is converted to abiraterone acetate or a salt thereof, wherein the improvement comprises an intermediate step of purifying the crude 3-β-acetoxyandrosta-5,16-diene-17-yl trifluoromethane sulphonate by crystallization from a solvent to obtain acetoxyandrosta-5,16-diene-17-yl trifluoromethane sulphonate as a crystalline solid. As discussed earlier, the triflate intermediate obtained in most of the prior art processes is isolated in crude form (mostly in form of oil) and is directly taken for next step without subjecting it to purification. The crude oil contains unreacted starting material DHEAA along with other impurities and decomposed products. The present invention provides a simple process of purification of 3-β-acetoxyandrosta-5,16-diene-17-yl trifluoromethane sulphonate by crystallizing it form a solvent without subjecting it to chromatographic purification. The process yields a solid triflate intermediate in purity greater than 98%. Handling of oily intermediate in commercial scale has disadvantages like difficulty in storage and transferring into different reaction vessels. Handling of solid in comparison to oil is much easier. The advantage becomes multifold when tonnage quantities of material is involved during commercial level production.

According to the invention, first step involves reacting dehydroepiandrosterone-3-acetate (DHEAA) with a triflating agent selected from triflic anhydride, triflic chloride or triflic acid.

The '766 patent has demonstrated that base having pKa of its conjugate acid less than 5.21 at 25° C. are not suitable for the reaction as they lead to more competing reactions causing complications. The '766 patent further states that reactions performed in ethers and hydrocarbons solvent showed problems with solubility of the reactant along with their reactivity and suggest that chlorinated solvents are the optimum solvents for the reaction.

In preferred embodiment of the improvement of the present invention in the first step DHEAA is triflated in the presence of a base in an aromatic hydrocarbon solvent. It was found that for a successful subsequent intermediate step of crystallization it was necessary to optimise the first step to form a crude 3-β-acetoxyandrosta-5,16-diene-17-yl trifluoromethane sulphonate which has amenable to crystallization and purification thereof. This optimization can be achieved by proper selection of conditions namely the base, the solvent and the ratio of the DHEAA to triflating agent. For example, suitable base for the first step included organic bases like triethylamine, diisopropylethyl amine, pyridine, 2,6-luitidine, N,N-dimethylaniline, N,N-diethylaniline, 2,6-di-tert-butyl-4-methylpyridine and the like. Contrary to the teachings of the '766 patent N,N-dimethylaniline with a pKa less than 5.2 could be suitably used in the first step and was a preferred base Suitable solvents included aromatic hydrocarbon solvent selected from toluene, xylene, mixtures thereof and the like. Present inventors have found that the first step can be optimized in order to form

In another preferred embodiment of the present improvement, the ratio of DHEAA:triflating agent is at least 1:2. More preferably, the ratio of DHEAA:triflating agent is in the range of 1:2 to 1:4. Most preferably, the ratio of DHEAA:triflating agent is 1:2.5.

In another preferred embodiment of the invention the solvent used in the intermediate crystallization step is a mixture of a water miscible organic solvent and water. More preferably, water miscible solvent is a ketone or an alcohol. Most preferably, ketone is acetone and the alcohol is isopropyl alcohol.

In another embodiment of the present improvement in the process for preparation of abiraterone acetate the improvement further comprises monitoring the unreacted DHEAA during the First step and obtaining and purifying crude 3-β-acetoxyandrosta-5,16-diene-17-yl trifluoromethane sulphonate after less than 5% DHEAA remains unreacted. We have surprisingly found that the ratio of DHEAA:triflating agent used in first step is an important factor to drive the reaction to a stage where more than 95% of DHEAA is consumed in the reaction. As per our findings, the ratio of DHEAA:triflating agent should at least be 1:2 for more than 95% conversion of DHEAA. As demonstrated in particular examples use of excess of triflic anhydride surprisingly provided desired crude 3-β-acetoxyandrosta-5,16-diene-17-yl trifluoromethane sulphonate that was amenable to crystallization. The consumption of DHEAA in the reaction can be measured by monitoring the reaction in frequent interval of time using techniques known to skilled artisan for instance Thin Layer Chromatography (TLC). Conversion of more than 95% of DHEAA obviates the need to recover the starting material

The preferred embodiments of the improvement of the present invention helps to attain faster reaction rate. The present improvement in the process enables production of commercial scale batch of the triflate intermediate in one shift of 24 h or less than that.

One of the major side reaction during the triflating step is elimination reaction which yields an impurity of a compound of Formula IV.

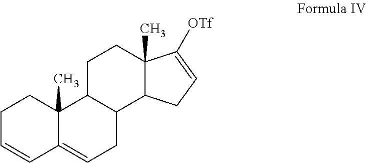

The impurity when carried forward in the next of coupling with diethyl-(3-pyridyl)-borane is converted into a triene impurity of Formula V.

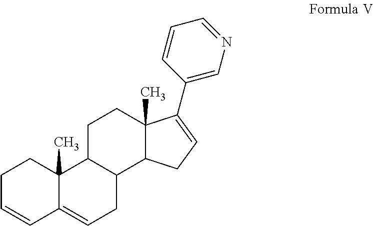

The recommended dose of ZYTIGA (Abiraterone acetate) is 1,000 mg (four 250 mg tablets) administered orally once daily is high. Hence, the content of impurity in the product are required to be maintained at very low level. Purification of the triflate intermediate by crystallization with solvent as provided by the present improvement yields a product with less than 0.10% of impurity of Formula IV. Preferably, the content of the impurity of compound of Formula IV in the triflate intermediate is less than 0.08%.

In another embodiment of the present improvement there is provided 3-β-Acetoxyandrosta-5,16-diene-17-yl trifluoromethane sulphonate in the form of a crystalline solid having a HPLC purity greater than 98% which is prepared by the improvement of the present invention. More preferably, the solid crystalline triflate intermediate have a HPLC purity of more than 99% and most preferably more than 99.5%.

The triflate intermediate thus formed can be converted into Abiraterone acetate or pharmaceutically acceptable salts thereof by the processes known in the art. For instance, the triflate intermediate is subjected to coupling with diethyl-(3-pyridyl)-borane in presence of a Palladium catalyst in a suitable solvent. Palladium catalyst can be Bis(triphenylphosphine)palladium(II) dichloride [Pd(PPh₃)₂Cl₂)] and suitable solvent can be tetrahydrofuran (THF).

Use of pure crystalline solid triflate intermediate for the preparation of abiraterone acetate enables the present inventors to use lesser quantity of diethyl-(3-pyridyl)-borane. For instance the '766 patent uses 1.7 molar equivalent of diethyl-(3-pyridyl)-borane with respect to the triflate intermediate whereas, the present improvement in the process enables to use only 1.05 molar equivalents. The discrete solid triflate intermediate with HPLC purity >97% and impurity of compound of Formula IV less than <0.5% has significant impact in achieving a crude Abiraterone acetate with HPLC purity greater than 90%.

The crude Abiraterone acetate obtained in second step can be purified by recrystallization successively from n-Heptane followed by Acetone:water (80:20). Alternatively, after work up, the reaction mixture is directly used, to prepare the p-TSA salt of Abiraterone acetate which further treated with base to yield abiraterone acetate having >99.5% purity by HPLC.

Abiraterone acetate or pharmaceutically acceptable salt thereof prepared by the improvement of the present invention has a HPLC purity of 99%, preferably more than 99.5% and most preferably more than 99.8%.

### GLOSSARY

### The Triflate Intermediate:

The triflate intermediate is defined as a compound of Formula III

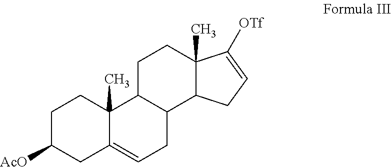

### Crude Abiraterone Acetate:

Crude Abiraterone acetate is the product formed in the reaction mixture when the triflate intermediate of Formula III is subjected to coupling with diethyl-(3-pyridyl)-borane in presence of palladium catalyst and/or isolated from the reaction mixture by any known technique under the purview of a skilled artisan like extracting the reaction mixture with a water immiscible solvent, washing it with water and concentrating the solvent, without carrying out any further purification by way of recrystallization, any chromatographic purification or the like.

### Crude Triflate Intermediate or Crude 3-β-Acetoxyandrosta-5,16-Diene-17-Yl Trifluoromethane Sulphonate:

The product formed in the reaction mixture when DHEAA is reacted with triflating agent and/or isolated from the reaction mixture by any known technique under the purview of a skilled artisan like quenching the reaction mixture in water and separating the organic layer, washing the organic layer with acid solution and concentrating the solvent, without carrying out any further purification by way of recrystallization, any chromatographic purification or the like.

### The Triene Impurity:

The triene impurity is a compound of Formula V

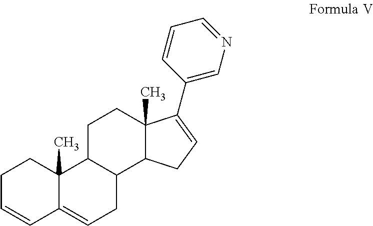

The present invention is further illustrated in detail with reference to the following example. It is desired that the example be considered in all respect as illustrative and are not intended to limit the scope of the claimed invention.

### Example 1

### Preparation of Triflate Derivative of Formula III

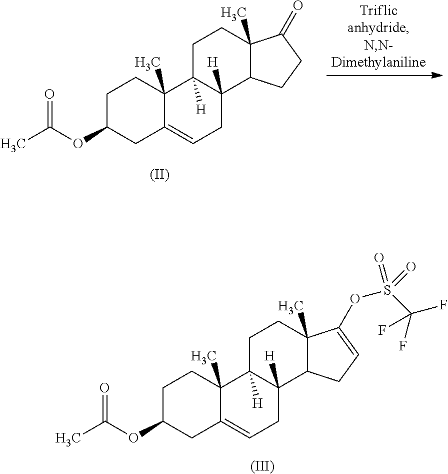

Triflic anhydride (213.43 g, 0.756 moles) was added to a stirred solution of Dehydroepiandrosterone acetate (II) (100 g, 0.3026 moles) in Toluene at 0-2° C. A solution of N,N-Dimethylaniline (82.5 g, 0.680) dissolved in Toluene was added over a period of 20-30 minutes at 3-7° C. Resulting reaction mixture was stirred for about 90 minutes at 3-7° C. After completion of reaction, water was added to quench the reaction and the biphasic solution was separated at room temperature. The product enriched organic layer was washed with 2N HCl followed by Brine solution. The product enriched organic layer was concentrated and degassed under reduced pressure below 45° C. The residual mass was dissolved in Acetone:Water mixture (80:20, 20 volumes) at 40-45° C., filtered hot to remove any suspended matter and the filtrate solution was cooled to −10 to −8° C. After maintaining the temperature for about 75 minutes the crystallized solid was filtered, washed with water and dried under vacuum below 40° C. to obtain the product in the form of a solid. (85 g). The crude product was dissolved in IPA:Water mixture (80:20, 15 volumes) at 70-80° C., treated with charcoal and cooled to 5-10° C. After maintain for 3-5 hours the product was filtered, washed with water and dried under reduced pressure below 45° C. to obtain pure (III). (yield 68 g 48.5% Th)
Description: Off white solid
Purity by HPLC: 99.7%

### Method of Analysis of the Triflate Intermediate Used in Examples Described Herein:

Buffer solution: Transfer 1.15 g of ammonium dihydrogen ortho phosphate in to a 1000 ml volumetric flask. Dissolve in and dilute up to mark with water. Adjust the pH of the solution to 3.00+0.10 with dilute ortho phosphoric acid solution.

Mobile phase-A: Mix buffer and acetonitrile in the ratio of 80:20. Filter through 0.22μ and degas prior to use.

Mobile phase-B: Mix buffer and acetonitrile in the ratio of 30:70. Filter through 0.22μ and degas prior to use.

Diluent: Mix water and acetonitrile in the ratio of 40:60. Filter through 0.22μ and degas prior to use.

Chromatographic system: Column: Acquity UPLC BEH C18 (100×2.1) mm, 1.7 μm

Flow rate: 0.4 ml/min.

Detector: 210 nm

Run time: 30 min.

Injection volume: 1.0 μl

Column temperature: 50° C.

### Example 2

### Preparation of Triflate Derivative of Formula III

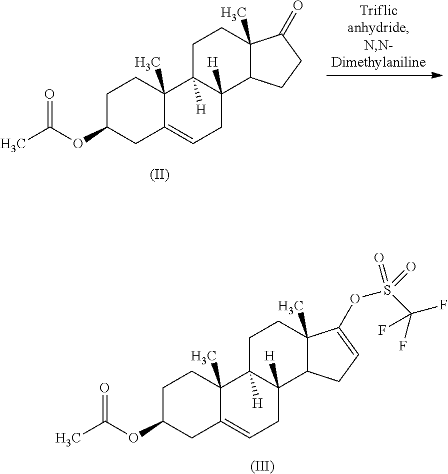

Triflic anhydride (32.01 kg) was added to a stirred solution of Dehydroepiandrosterone acetate (II) (15 kg) in Toluene at 0-2° C. A solution of N,N-Dimethylaniline (12.37 kg) dissolved in Toluene was added over a period of 20-30 minutes at 3-7° C. Resulting reaction mixture was stirred for about 90 minutes at 3-7° C. After completion of reaction water was added to quench the reaction and the layers were separated at room temperature. The product enriched organic layer was washed with 2N HCl followed by brine solution. The product enriched organic layer was concentrated and degassed under reduced pressure below 45° C. The residual mass was dissolved in Acetone:Water mixture (80:20, 20 volumes) at 40-45° C. The solution was cooled to −10 to −8° C. After maintaining the temperature for about 75 minutes the solid was filtered, washed with water and dried under vacuum below 40° C. to obtain the product in the form of a solid. (12.8 kg). The crude product was dissolved in IPA:Water mixture (80:20, 15 volumes) at 70-80° C., treated with charcoal and cooled to 5-10° C. After maintain for 3-5 hours the product was filtered, washed with water and dried under reduced pressure below 45° C. to obtain pure (III). (Yield; 10.4 kg, 49.5%)
Description: Off white solid.
Purity by HPLC: 98.5%

### Example 3

### Preparation of compound of Formula I

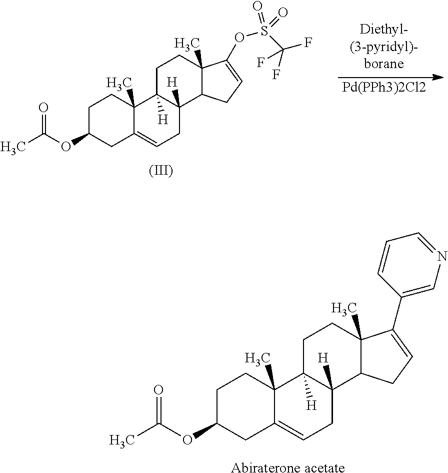

Diethyl(3-pyridyl)borane (35 g) was added to a stirred solution of Compound of formula (III) (100 g, 0.216 moles) in THF (800 ml) containing Pd(PPh₃)₂Cl₂ catalyst (0.6 g, 0.00085 moles). An aqueous solution of sodium carbonate (82.4 g, 0.778 moles) was then added and the reaction mixture was heated to reflux temperature (about 65° C.) under stirring. Maintained the reaction for 4 hours and its completion was checked by TLC. The reaction mixture was cooled to room temperature and partitioned between Ethylacetate (400 ml) and water. The organic layer was washed thrice with brine solution and concentrated under reduced pressure at 45-55° C. The degassed oily mass was stirred in Methanol (800 ml) and was heated at 45-55° C. after adding charcoal to it. The precipitated solid along with charcoal was filtered and washed with Methanol (200 ml). Water (450 ml) was slowly added to the filtrate at 40-50° C. under stirring. The resulting suspension was maintained at 20-25° C. for 3 hours and filtered the solid. Filtered solid was washed with water and dried under reduced pressure at 55-65° C. to obtain crude Abiraterone acetate.
Yield: 80 g, 95% Th.
Chromatographic (HPLC) purity 95.7%

Obtained Abiraterone acetate further purified by direct purification using heptane or optionally using salt screen purification as described in specification.

### Example 4

### Direct Purification Using Heptane

Crude Abiraterone acetate (40 g) was dissolved in n-Heptane (1200 ml) at 75-80° C. and added charcoal to it. Charcoal was filtered at same temperature and the filtrate was concentrated to about 50%. The suspension was cooled to 20-25° C. and maintained for 5 hours. Filtered the solid, washed with n-Heptane and suck dried it. The solid was dissolved in a mixture of Acetone:Water (80:20, 10 volumes) at 55-65° C. and the solution was gradually cooled to 15-20° C. After maintaining for 8 hours the solid was filtered, washed with water and dried under reduced pressure at 55-65° C. to obtain pure Abiraterone acetate (I).
Yield: 26.8 g
Chromatographic (HPLC) purity: 99.91%

### Example 5

### Optional Purification by Salt Screening

p-Toluenesulfonic acid (20.4 g) was added to a stirred solution of crude Abiraterone acetate (40 g) dissolved in Toluene (400 ml) at 15-20° C. and maintained for 15-30 minutes. Added Hexane (400 ml) to it and stirred the suspension for 45-60 minutes. Filtered the solid and washed with hexane. Product cake was suck dried under nitrogen atmosphere. The p-TSA salt was suspended in water and Toluene mixture and treated with sodium bicarbonate. The product enriched organic layer was washed with brine solution followed by treatment of Anhydrous sodium sulphate and charcoal. The filtrate was concentrated under reduced pressure and after degassing a mixture of Acetone:Water (80:20) was added. A mixture was heated at 55-65° C. and gradually cooled to 15-20° C. After maintaining for 8 hours the solid was filtered, washed with water and dried under reduced pressure at 55-65° C. to obtain pure Abiraterone acetate (I).
Yield: 27 g
Chromatographic (HPLC) purity 99.82%

1. An improvement in the process of preparation of abiraterone acetate or a pharmaceutically acceptable salt thereof which process comprises a first step in which dehydroepiandrosterone-3-acetate (DHEAA) is triflated in the presence of a base and a solvent to form crude 3-β-acetoxyandrosta-5,16-diene-17-yl trifluoromethane sulphonate and a second step wherein the crude 3-β-acetoxyandrosta-5,16-diene-17-yl trifluoromethane sulphonate is converted to abiraterone acetate or a salt thereof, wherein the improvement comprises an intermediate step of purifying the crude 3-β-acetoxyandrosta-5,16-diene-17-yl trifluoromethane sulphonate by crystallization from a solvent to obtain acetoxyandrosta-5,16-diene-17-yl trifluoromethane sulphonate as a crystalline solid.

2. The improvement as in claim 1 wherein the improvement further comprises monitoring the unreacted DHEAA during the first step and obtaining and purifying crude 3-β-acetoxyandrosta-5,16-diene-17-yl trifluoromethane sulphonate after less than 5% DHEAA remains unreacted.

3. The improvement as in claim 2 wherein in the first step DHEAA is triflated in the presence of a base in an aromatic hydrocarbon solvent

4. The improvement as in claim 3 wherein the ratio of DHEAA:triflating agent is at least 1:2

5. The improvement as in claim 3 wherein the base is N,N-Dimethylaniline

6. The improvement as in claim 1 wherein the solvent used in the intermediate step is a mixture of a water miscible organic solvent and water.

7. The improvement as in claim 6 wherein the water miscible solvent is a ketone or an alcohol

8. The improvement as in claim 7 wherein the ketone is acetone and the alcohol is isopropyl alcohol.

9. The improvement as in claim 1 wherein improvement further comprises
a. Triflating DHEAA using a ratio of DHEAA:triflating agent of at least 1:2, in the presence of N,N-Dimethylaniline in toluene;
b. monitoring unreacted DHEAA during triflation and obtaining and purifying crude 3-β-acetoxyandrosta-5,16-diene-17-yl trifluoromethane sulphonate after less than 5% DHEAA remains unreacted;
c. the ratio of DHEAA:triflating agent is at least 1:2; and
d. purifying the crude 3-β-acetoxyandrosta-5,16-diene-17-yl trifluoromethane sulphonate by crystallization from a solvent is a mixture of water and acetone or isopropyl alcohol.

10. 3-β-Acetoxyandrosta-5,16-diene-17-yl trifluoromethane sulphonate in the form of a crystalline solid having a HPLC purity greater than 98% obtained by the improvement as claimed in claim 1.
